(19) United States
(12) Patent Application Publication
(10) Pub. No.: US 2022/0189635 A1
(43) Pub. Date: Jun. 16, 2022

(54) SYSTEM AND METHOD OF ASSISTING MEDICAL TREATMENT

(71) Applicant: Dishahealth.AI, Inc., San Francisco, CA (US)

(72) Inventors: Ravi Gopalakrishnan, San Francisco, CA (US); Jiji Nair, Santa Clara, CA (US)

(21) Appl. No.: 17/549,801

(22) Filed: Dec. 13, 2021

Related U.S. Application Data
(60) Provisional application No. 63/125,845, filed on Dec. 15, 2020.

(51) Int. Cl. G16H 50/20; G16H 70/20; G16H 70/40; G16H 10/60

(52) U.S. Cl. CPC G16H 50/20; G16H 10/60; G16H 70/40; G16H 70/20

(57) ABSTRACT

A system and method of assisting medical treatment decisions, are described. The method includes receiving unstructured data, including real world data, scientific document data, and expert knowledge data, corresponding to patient journeys. The method includes generating unstructured data from the structured data, including expert defined rules based on the expert knowledge data. The method includes determining treatment pattern data based on the structured data. The treatment pattern data can include treatment patterns used by a physician to treat a population, and may be transmitted for display at a client device to assist in making medical treatment decisions. Other embodiments are also described and claimed.

This application claims the benefit of priority of U.S. Provisional Patent Application No. 63/125,845, filed on Dec. 15, 2020, which is incorporated herein by reference in its entirety to provide continuity of disclosure.

### BACKGROUND

### Field

The present disclosure relates to the field of software platforms and more particularly, to software platforms in the medical field.

### Background Information

Medical treatment decisions have multiple dimensions, including: patient characteristics; physician education, physician beliefs and behaviors; standards, guidelines and clinical pathways for treatment; health system guidelines, and payer guidelines. This makes such decisions very complex, and the complexity is compounded by rapid advances in science, e.g., the accelerating discovery of specialty drugs and personalized medicine, that physicians are hard-pressed to research, let alone integrate, into their daily practice. In practice, a universal approach to deciding how a patient gets access to appropriate medicines at appropriate times may be used. Such an approach may, however, cause many patients to be left behind by the advancing science. In some circumstances, the universal, non-personalized approach, can lead to poor patient outcomes, increased costs for all stakeholders in the medical treatment, and loss of revenue for pharmaceutical companies that could otherwise provide more appropriate drugs and medicines.

### SUMMARY

Existing systems used to assist in making medical treatment decisions report raw clinical data associated with patient treatments. Such raw data reporting, however, fails to explain or infer physician intent for a treatment decision. Nor does such raw data reporting capture the beliefs and behaviors of the physician making the treatment decision, or the patient response to the treatment decision. Since the number of historical raw clinical events is so large, it is currently very difficult for a human to extract from the reported data the physician intent, beliefs, or behaviors. Accordingly, there is a need to leverage advances in machine intelligence, and combine such machine intelligence with expert knowledge to provide an explainable summary of treatment plans and patterns that will allow users to understand, from the reported data, the physician intent, beliefs, or behaviors.

A method of assisting medical treatment decisions is provided herein. A computing system configured to perform the method, and non-transitory computer readable mediums storing instructions executable by the computing system to cause the computing system to perform the method, are also provided herein. In an embodiment, the method includes receiving unstructured data from various sources, which can describe patient journeys. For example, the unstructured data can include real world data, scientific document data, and expert knowledge data corresponding to the patient journeys. Unstructured data can be generated based on the structured data. For example, expert defined rules can be generated based on the expert knowledge data. Using the structured data, information about a particular patient or physician can be determined. For example, treatment pattern data can be determined based on the structured data. The treatment pattern data can correspond to a treatment pattern used by the a physician to treat a patient population. Accordingly, the treatment behavior and/or beliefs of the physician can be transmitted for display as a recommendation.

Other information can be determined and transmitted for display, based on the unstructured and structured data. For example, a search query can include patient criteria, and historical outcomes of similar patients treated with a treatment pattern can be determined and matched to the query. A treatment recommendation, e.g., a treatment pattern that can benefit the patient matching similar patient profiles, can be transmitted for display as a recommendation.

Search queries can be received from digital assistant applications running on remote client devices, and responsive search results can be transmitted to the remote client devices for display to users, e.g., representative of pharmaceutical companies, physicians, etc. In an embodiment, the treatment pattern includes one or more treatment blocks, which can include respective sequences of treatment activity. Accordingly, the treatment pattern can be displayed as a sequence of treatment blocks, e.g., interconnected by visual connectors. The treatment pattern visualization and other visualizations can be used to output treatment recommendations to users, as well as to allow users to interact with the system to enter useful data, e.g., the expert knowledge data, that can be used to further refine results and make recommendations.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings.

FIG. 1 is a model of an example ecosystem for providing medical treatment, in accordance with an embodiment.

FIG. 2 is a model of an example approach to educating and communicating to assist in making medical treatment decisions for providing medical treatment to a patient, in accordance with an embodiment.

FIG. 3 is a block diagram of an example computing environment for assisting medical treatment decisions, in accordance with an embodiment.

FIG. 4 is a flowchart of a method of assisting medical treatment decisions, in accordance with an embodiment.

FIG. 5 is a model of a treatment assistance platform, in accordance with an embodiment.

FIG. 6 is a model of real world data, in accordance with an embodiment.

FIG. 7 is a pictorial view of expert knowledge being captured in a treatment assistance platform, in accordance with an embodiment.

FIG. 8 is a table of treatment pattern data generated by a treatment assistance platform, in accordance with an embodiment.

FIG. 9 is a pictorial view of a user interface visualization of diagnosed patients at a national level, in accordance with an embodiment.

FIG. 10 is a pictorial view of a user interface visualization of diagnosed patients at a state and local level, in accordance with an embodiment.

FIG. 11 is a pictorial view of a user interface visualization of a payer view for diagnosed patients, in accordance with an embodiment.

FIG. 12 is a flowchart of a method of searching for visualizations to assist medical treatment decisions, in accordance with an embodiment.

FIG. 13 is a pictorial view of a physician summary view of a user interface, in accordance with an embodiment.

FIG. 14 is a pictorial view of a treatment pattern view of a user interface, in accordance with an embodiment.

FIG. 15 is a pictorial view of a patient funnel selector of a user interface, in accordance with an embodiment.

FIG. 16 is a pictorial view of a treatment plan summary of a user interface, in accordance with an embodiment.

FIG. 17 is a pictorial view of a patient journey summary of a user interface, in accordance with an embodiment.

FIG. 18 is a pictorial view of a patient journey timeline visualization of a user interface, in accordance with an embodiment.

FIG. 19 is a block diagram of an example computing device that may perform one or more of the operations described herein, according to some embodiments.

### DETAILED DESCRIPTION

Embodiments describe a system and method of assisting medical treatment decisions. The system and method can be used in assisting medical treatment decisions related to treating patient diagnosed with cancer, for example. However, the system and method may also be used to assist treatment of other diseases or conditions, such as asthma. Thus, reference to the system as being used for any particular treatment is not limiting.

In an aspect, a system and method of assisting medical treatment decisions is provided. The system and method can be embodied in a treatment assistance platform that provides real-time insights on patient treatments and physician education. The system and method leverage real world data, scientific and research documents, and expert knowledge, and use machine intelligence to generate and visualize explainable treatment plans at a patient level, as well as summarize treatment patterns at a physician, health system, and payer level. The visualizations provide deeper insights into physician education gaps, knowledge levels, beliefs and behaviors.

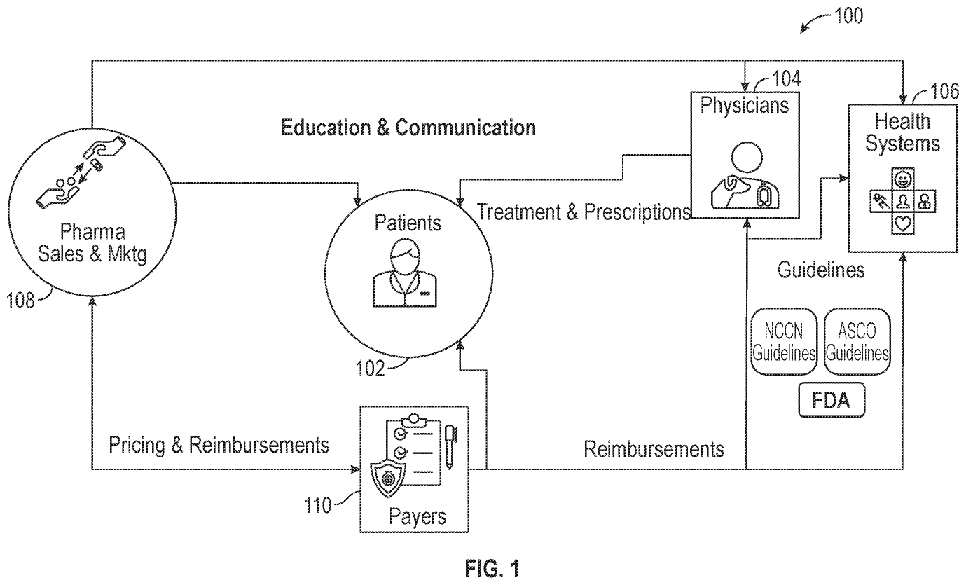

Referring to FIG. 1, a model of an example ecosystem for providing medical treatment is shown in accordance with an embodiment. The medical treatment ecosystem 100 includes several stakeholders that interact to provide treatment to a patient 102 (or a group of patients). Stakeholders include a physician 104 (or a group of physicians) that treats the patient 102. The physician(s) 104 interact with representatives of health systems 106, e.g., hospitals and hospital administrators, as well as representatives of pharmaceutical companies 108, e.g., sales and marketing representatives, to make appropriate decisions about treating the patient 102. For example, pharmaceutical representatives can communicate with the physician 104 and/or the patient 102 to provide education and recommendations regarding treatments that could benefit the patient 102 based on the particular characteristics of the patient. Such characteristics can include co-morbidities, genetic mutations, and other attributes that medical experts within the pharmaceutical company 108 have determined as being indicative that the patient 102 can benefit from the treatments. Similarly, the pharmaceutical company 108 can educate the health systems 106 as well as representatives of payers 110, e.g., insurance company representatives, to help the market move toward valuable treatments and to efficiently price those treatments.

Historically, pharmaceutical companies use customer relationship management (CRM) systems and tools to educate and communicate with physicians 104, patients 102, health systems 106, and payers 110. The CRM systems are typically accessed by representatives of the pharmaceutical company 108 who are insulated from each other, and who communicate separately with counterparts in the ecosystem. For example, a marketing representative, a sales representative, and a medical expert from the pharmaceutical company 108 may each communicate individually with the physician 104 and the patient 102. Similarly, an account representative may communicate with the health system 106, and a market access representative may communicate with the payer 110. The communications are siloed and are part of a “one size fits all” approach because existing CRM systems do not have access to real-time insights about the patient characteristics or the physician treatment patterns, which would otherwise allow for personalized treatments to be recommended to benefit the patient 102. More particularly, while existing CRM systems may contain a mass of raw data about treatments, they cannot be used to efficiently and accurately determine and provide real-time insights about patients and physician education, knowledge, beliefs and behaviors, which may influence treatment decisions on a per-patient basis.

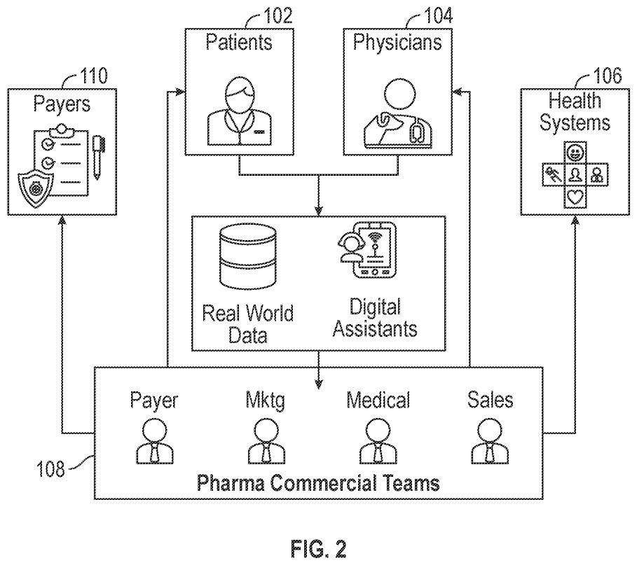

Referring to FIG. 2, a model of an example approach to educating and communicating to assist medical treatment decisions for providing medical treatment to a patient is shown in accordance with an embodiment. The embodiments described herein fill the gap left by existing CRM systems. Rather than follow a “one-size-fits-all” approach, the systems and methods described below follow a patient-centered, data-driven, personalized approach to providing education and communication. This approach, rather than being siloed, is coordinated and integrated in that information about the patient 102 and the physician 104 flow to the system, which may be used by the pharmaceutical company 108 to develop insights about the patient characteristics or the physician treatment patterns. Such information can be provided as real-world data collected from electronic medical record (EMR) systems, or entered through client devices used by the patient 102 or the physician 104. This coordinated information flow can allow the pharmaceutical company 108 representatives to communicate targeted treatment options that are personalized to the patient 102 or the physician 104. For that matter, the system itself can be used by the physician 104, the patient 102, the health system 106, or the payer 110 to access the insights that are most helpful to any of those entities. More particularly, the members of the ecosystem can be engaged through marketing channels or directly by the treatment assistance platform described below to help make the right treatment decision for the patient 102. Accordingly, the system and method enables precision targeting at scale and in a personalized manner to ensure that patients receive proper, individualized treatment.

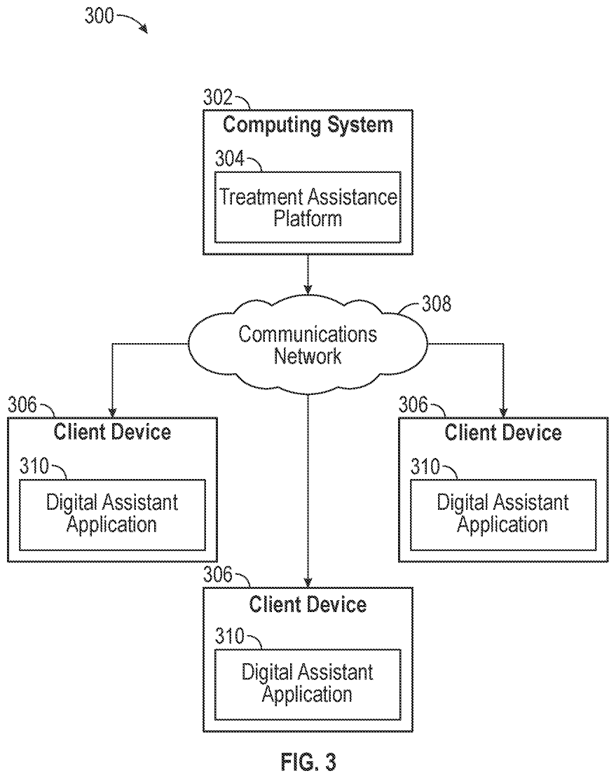

Referring to FIG. 3, a block diagram of an example computing environment for assisting medical treatment decisions is shown in accordance with an embodiment. As shown, the environment 300 can include one or more computing systems. For example, the environment 300 can include a computing system 302 running a treatment assistance platform 304. The computing system 302 can be interconnected with one or more remote client devices 306 (that may also be computing systems) via a communications network 308. The communications network 308 may be the internet, a wide area network (WAN), intranet, or other suitable network. Each client device 306 may be executing a digital assistant application 310. The digital assistant application 310 can be a computer-based software application or a web application. For example, the digital assistant application 310 can be integrated within a CRM application or an EMR application, e.g., as a flashcard, or as a standalone web application. The digital assistant application 310 can communicate with, or form a portion of, the treatment assistance platform 304. For example, the client devices 306 may be used by the physician 104, the payer 110, or the health system 106 to receive data from, or send data to, the computing system 302 that could be under the control of the pharmaceutical company 108. This distribution of computing systems 302 amongst the various stakeholders is provided by way of example, however, and it will be appreciated that the computing system 302 may be viewed as a distributed computing network having several devices under the control of the ecosystem members, and running all or a portion of the treatment system platform while interconnected by the communications network 308. Accordingly, the solution can be integrated at a point of care, in a pharmaceutical company CRM system, or other locations.

The computing system 302 may be hosted on one or more local servers, may be a cloud based system, or may be a hybrid system with local servers and in the cloud. The computing system 302 is maintained by engineers which develop the treatment assistance platform 304. Although FIG. 3 shows only a select number of computing devices and/or systems, the environment 300 may include any number of computing devices and/or systems that are interconnected in any arrangement to facilitate the exchange of data between the computing devices and/or systems.

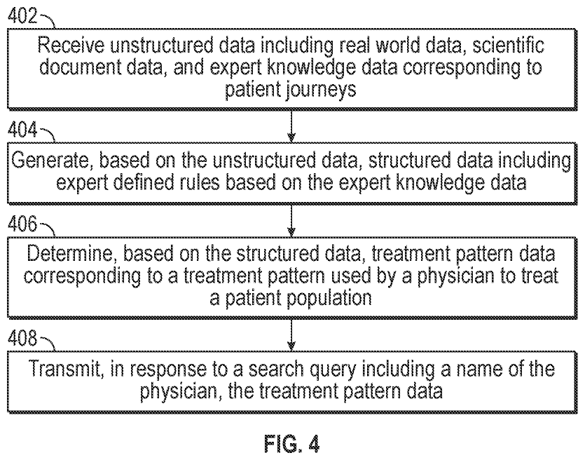

Referring to FIG. 4, a flowchart of a method of assisting medical treatment decisions is shown in accordance with an embodiment. The method may be performed by the system described below, and thus, the operations of the method will be described in combination with the system description below. The system and method advantageously aggregates scientific information, patient longitudinal data, and physician historical treatment data, and combines that data with expert scientific knowledge to provide real-time recommendations of treatments that will benefit the particular patient and integrate well with the deduced beliefs and behaviors of the physician. In essence, the system and method provide a real-time suggestion for treatment that is personalized to both the patient and the physician.

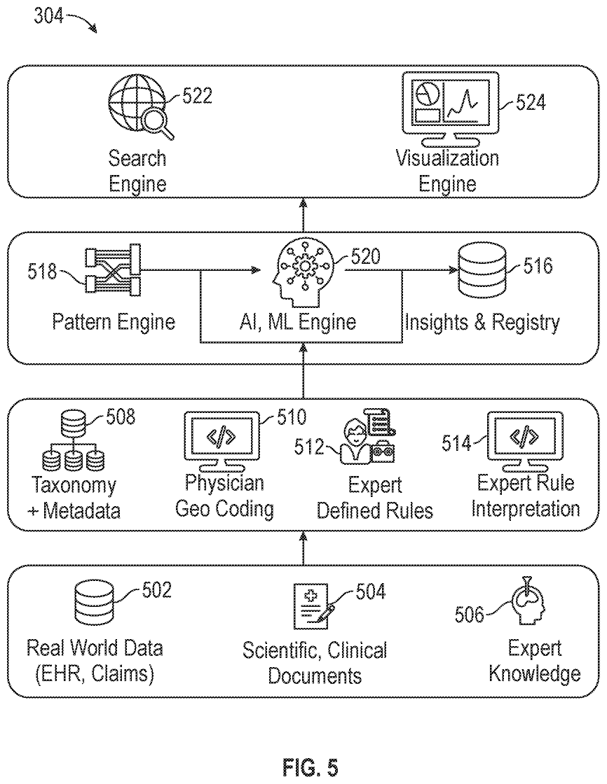

Referring to FIG. 5, a model of a treatment assistance platform is shown in accordance with an embodiment. At operation 402, unstructured data is received by the treatment assistance platform 304. The unstructured data can be collected by data aggregators, such as data aggregators for insurance claims, data aggregators of EMR platforms, or data aggregators in communication with other data sources. The unstructured data may also be entered by stakeholders of the ecosystem. For example, medical representatives of the pharmaceutical company 108 may annotate data through the digital assistant application 310, as described below, or physicians may enter case notes into the digital assistant applications 310. The entered data can be aggregated as unstructured data for use by the treatment assistance platform 304.

In an embodiment, the unstructured data includes real-world data 502. The real-world data 502 can include information about a patient journey, e.g., a longitudinal view of the treatment of the patient from an initial diagnosis to a current stage of disease or treatment. The real-world data 502 can include information about sequences of medically relevant events along the patient journey, gathered from various sources.

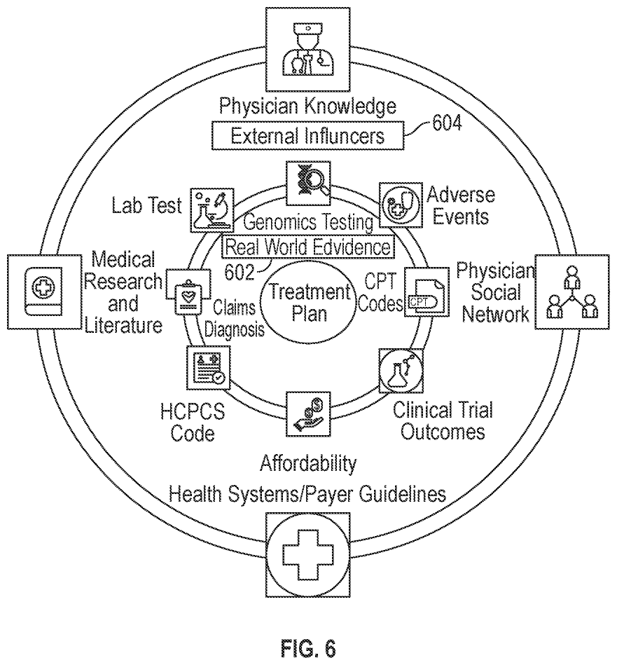

Referring to FIG. 6, a model of real-world data is shown in accordance with an embodiment. Sources of real-world data 502 include real-world evidence 602. The real-world evidence 602 can include data collected from EMR records. For example, the EMR records can indicate adverse events from patient journeys. The real word evidence can include insurance claims. For example, the insurance claims can be used to determine claims diagnosis, healthcare common procedure coding system (HCPCS) codes, current procedural terminology (CPT) codes, etc. The real-world evidence 602 can include pharmacy data. For example, pharmacy data can include clinical trial outcomes. The real-world evidence 602 can include pathology and genetic labs. For example, the real-world evidence 602 can include lab test results, genomics testing results, imaging data from physician visits, etc. The real-world evidence 602 can include information from health systems 106. For example, the real-world evidence 602 can include affordability data collected from health care administrators.

The real-world data 502 can include external influence data 604, e.g., information about or from external influencers. For example, physician knowledge can be included in the real-world data 502. Information about health systems and payer guidelines may be aggregated in the real-world data 502. Similarly, medical research and literature can be collected and aggregated. External influence data 604 may also include physician social network information, e.g., social connections between different physicians that may indicate an influence of those physicians on each other.

Referring again to FIG. 5, in an embodiment, the unstructured data includes scientific document data 504 corresponding to patient journeys. Scientific document data 504 can include scientific, clinical, and research documents and guidelines. For example, scientific and clinical documents may be documents from a website of the pharmaceutical company 108. Such documents could include a collection of documents that are used to license or sell approved drugs. The scientific document data 504 could also include publications discussing disease, clinical study findings, etc. An example of such medical research publications includes data that can be collected and aggregated from medical research repositories or databases, such as PubMed®. In an embodiment, the scientific document data 504 includes clinical trial data aggregated from clinical trial registry sources such as clinicaltrials.gov.

In an embodiment, the unstructured data includes expert knowledge data 506 corresponding to patient journeys. The expert knowledge data 506 can be collected or entered by scientific researchers, e.g., medical representatives of the pharmaceutical company 108. Alternatively, the expert knowledge 506 can come from physicians, payers, etc. As an example, the expert knowledge data 506 can include disease staging and treatment information, from an expert point of view. Such expert scientific knowledge can be immense and distributed throughout the ecosystem, and may not be easily disseminated to physicians through traditional channels. More particularly, it can be difficult for physicians to keep up with the advances in science as they are discovered by, e.g., pharmaceutical companies, and to understand how to treat patients with such advances. The treatment assistance platform 304 allows such information to be aggregated and disseminated through targeted recommendations, as described below.

By way of example, expert knowledge data 506 can include a document summarizing breast cancer staging and treatment. The document can describe treatment modalities, such as surgery, radiation therapy, chemotherapy, hormone therapy, targeted therapy, etc. The document can describe disease staging, e.g., Stage I through Stage IV of breast cancer, and associate recommended treatments with each stage. The document may therefore summarize, from an expert point of view, disease staging and treatment information that can be useful in recommending treatment of patients.

Expert knowledge data 506 may encompass other annotations or collected information that are relevant to treatment. For example, the expert knowledge data 506 may include one or more of drug indication data or patient response data. The drug indication data can include indications of drug approval. More particularly, the data can describe a disease that a drug is approved to treat. The patient response data can include information about how a certain patient or patient population is likely to respond to a drug. For example, the patient population may have a genetic mutation that causes a response to the drug, and that information can be collected by the treatment assistance platform 304.

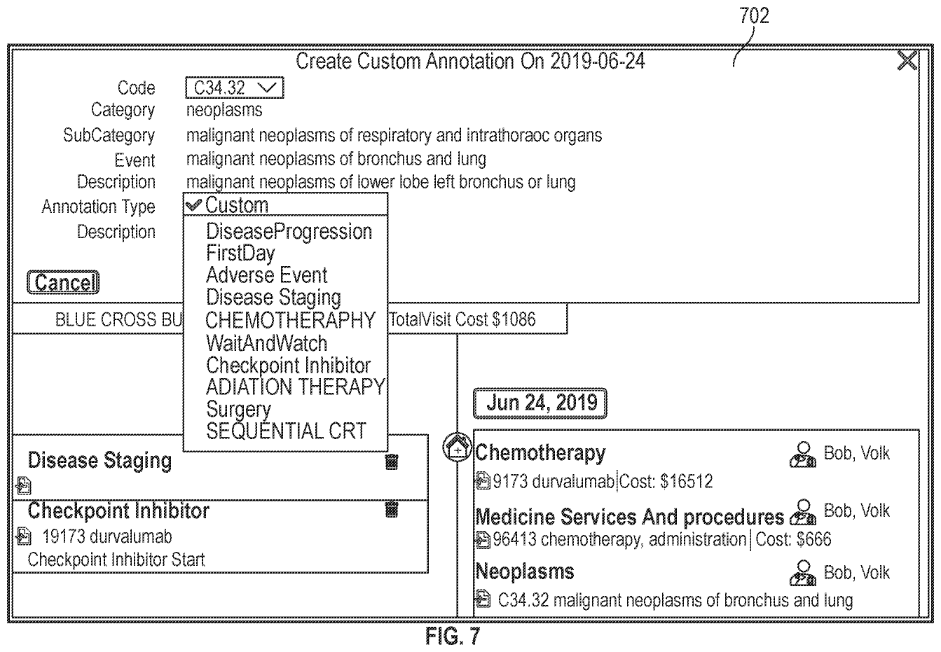

Referring to FIG. 7, a pictorial view of expert knowledge being captured in a treatment assistance platform is shown in accordance with an embodiment. Expert knowledge can be captured through annotations and labeling of patient journey data. For example, a medical representative of the pharmaceutical company 108 can use the treatment assistance platform 304 to visualize a patient journey, e.g., from a patient treated by a particular physician, and to create annotations that are aggregated as expert knowledge data 506. As described below, the expert can have multiple views into the patient journey. These visualizations may allow the expert to pinpoint, based on their knowledge of disease staging and treatment, the reasons behind treatment decisions that were made by the physician. The expert can create custom annotations 702 through a user interface of the treatment assistance platform to capture these inferences. For example, the custom annotation 702 can allow the expert to enter a code, category, subcategory, event, or description to summarize how a treatment decision made by the physician fits within a logical framework of preferred treatment decisions according to current scientific advances. The platform may also allow an expert to use high-level language to explain a treatment scenario.

At operation 404, one or more processors of the computing system 302 can generate structured data based on the unstructured data. As described above, the unstructured data can combine real-world data, scientific documents, and expert knowledge. The expert knowledge, e.g., of a scientific expert, clinical advisor, etc., is encoded to capture scientific advances as well as patient journeys that are based on physician decisions. By combining the various data, the treatment assistance platform 304 can discover physician prescribing behaviors, treatment patterns, and other valuable information that allows for personalized suggestions to be made that conform to the physician's attitudes. For example, the structured data can be used to determine treatment recommendations that parallel the apparent preferences of the physician to particular drug prescriptions.

The structured data can include layers of information, which may be specific to a particular disease. In an embodiment, the real-world data 502 is processed and transformed into a common format for machine learning. The real-world data 502 and the scientific document data 504 can be processed by machine learning classifiers to generate taxonomical metadata 508. For example, the unstructured data can be tagged by machine learning classifiers with medical-specific topics, such as inhibitors or proteins that cause genetic mutations, mechanism of action, safety profile of a drug, etc.

In an embodiment, the real-world data 502 and/or the scientific document data 504 are mined to generate multi-category taxonomy data. For example, scientific and clinical documents of the scientific document data 504 can be mined to generate multi-category taxonomy data with associated metadata. In one embodiment, such metadata may include tagging claim codes to a specific diagnosis, procedure, drug name, adverse event, etc.

Multi-category taxonomy data may be three-level taxonomy data. More particularly, the taxonomy can include category, sub-category, and event metadata. The metadata may, however, include additional levels, e.g., code and description metadata. By way of example, an insurance claim may be processed to generate taxonomical metadata 508 including a category (neoplasms), sub-category (malignant neoplasms of respiratory and intrathoracic organs), event (malignant neoplasm of bronchus and lung), code (C34.32), and description (malignant neoplasm of lower lobe left bronchus or lung) metadata. This metadata can be for real world medical datasets, e.g., from diagnostic information, HCPSC procedures, CPT codes, pharmacy prescriptions, lab test codes, imaging test codes, genetic testing data, etc. The taxonomy data can explain claims codes, for example, in the context of treatment during the patient journey.

The structured data can include geocode data 510. The geocode data 510 can geocode the real-world data 502 at a physician level. For example, locations of treatment activity can be geocoded using location information, e.g., state, city, address, in the real-world data 502. The geocode data 510 can be used to generate and display visualizations of treatment activity. As described below, visualizations can include the display of diagnosed patients at a national, state, or local level.

In an embodiment, the structured data includes expert define rules 512, which are based on the expert knowledge data 506. High-level declarative language or treatment plan blocks are used to convert the expert knowledge into the expert define rules 512. For example, an English-like language, e.g., “show me all patients whose disease progressed following concurrent chemo/radiation within six months,” is used to query claims, lab results, medical imaging data, etc. The query can be made without understanding the complexity and structure of the data. Accordingly, declarative language can capture expert define rules 512.

The treatment assistance platform 304 can generate expert rule interpretation data 514. More particularly, the expert knowledge data 506 can be processed and interpreted to generate associated derived data. For example, the platform may map words to associate disease context and treatment conditions and use artificial intelligence to convert the context and criteria to a structured query language (SQL) query. The interpreter engine can translate, for example, a treatment scenario described by an expert using high-level language into a query that can be used to identify matching patients who fulfill given criteria. The derived data can be stored as, or used to generate, insights and registry data 516.

The treatment assistance platform 304 may further generate a pattern engine 518. The pattern engine 518 can form building blocks of patient level treatment and physical level treatment patterns. More particularly, the pattern engine 518 of the treatment assistance platform 304 can encode a summary of treatment plans and patterns at a patient and/or physician level.

Data generated by the pattern engine 518 can be stored as insights and registry data 516. Alternatively, data generated by the pattern engine 518 can be used by a machine intelligence engine 520 to segment and cluster patients and physicians based on similarity, and also make predictions on patient outcomes and physician behaviors. The machine intelligence engine 520 can include software and use-case driven algorithms trained on raw and derived data to achieve this.

In an embodiment, the machine intelligence engine 520 includes machine learning, deep learning, recurrent neural network, or other machine intelligence. The type of machine intelligence algorithm may be driven by a specific use case, and a suitable choice may be selected from a library for the use case being solved for. In one embodiment, segmentation and clustering are case driven. For example, the machine intelligence engine 520 might segment physicians within similar beliefs but cluster them based on treatment patterns. The machine intelligence engine 520 may also predict that a patient outcome could include severe sides effects for a therapy decision, which includes risk of hospitalization, risk of death, etc. In such case, a prediction model may be trained using a variety of features from the aggregate events of patients partitioned on a positive and negative data set. The clusters and segment representation of patients and physicians and predictive insights on patients and physicians can be added to the insights and registry data 516.

The data generated by the pattern engine 518 and the machine intelligence engine 520 covers the patient journey of the patient. More particularly, the unstructured and structured data that the insights and registry data 516 is based on goes back in time, e.g., to a start of an initial diagnosis of a patient, and thus can cover the entire patient journey. In an embodiment, the insights and registry data 516 groups a sequence of medically relevant events to summarize and explain a block of a treatment plan using the expert define rules 512. More particularly, the insights and registry data 516 can explain treatment blocks, which are blocks of treatment that describe a sequence of treatment activity taken during the treatment of a patient. The insights and registry data 516 can be a secondary dataset to augment raw data with expert define rules 512.

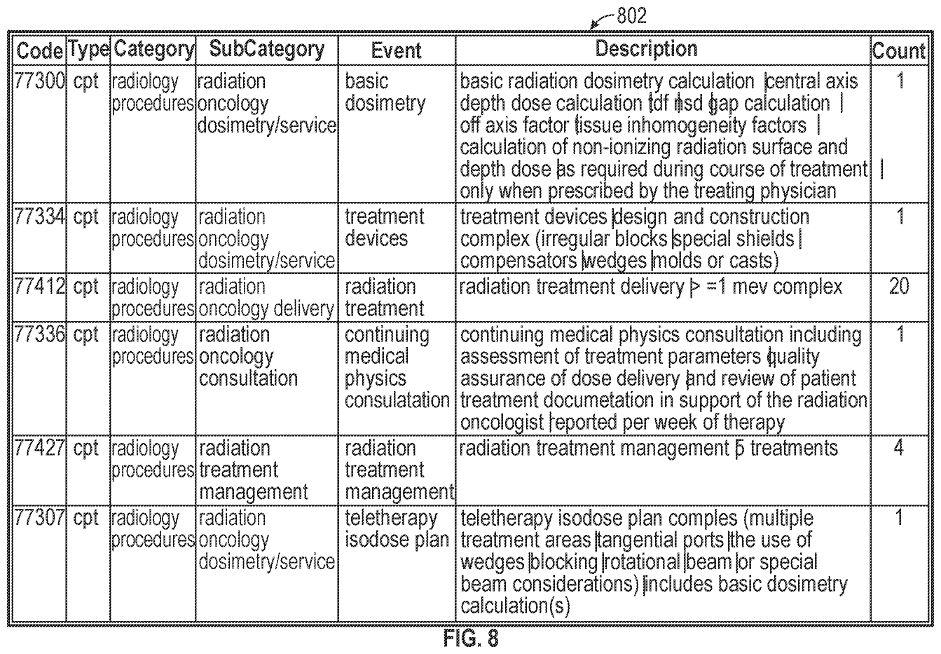

Referring to FIG. 8, a table of treatment pattern data generated by the treatment assistance platform is shown in accordance with an embodiment. At operation 406, the treatment assistance platform 304 can determine treatment pattern data 802 corresponding to a treatment pattern used by a physician. The treatment pattern may be used by the physician to treat a patient population. For example, the treatment pattern can include one or more treatment blocks that are used by the physician in the course of treating patients. The treatment assistance platform 304 can generate the treatment pattern data 802 by looking at the time sequence of events within the context of treatment. For example, the example illustrated in FIG. 8 shows a treatment pattern of radiation therapy for cancer patients. The treatment pattern data 802, which can be generated utilizing the expert define rules 512 and expert rule interpretation can be used to create patient journey blocks. For example, as shown in the table, radiation therapy can include a sequence of events that includes preparatory treatment, dosimetry, and periodic analysis to determine patient response and anticipatory treatment of side effects. The treatment assistance platform 304 can alternatively summarize and explain treatment blocks that include disease staging, disease progression, disease free interval, wait and watch, adverse events, etc. Any such treatment blocks can be determined and stored as insights and registry data 516 that can be used to determine treatment patterns of physicians.

It will be appreciated that the treatment blocks that are determined and stored as insights and registry data 516 can represent the entire patient journey of several, e.g., many, patients as well as the treatment patterns that are used by several, e.g., many physicians. The data may therefore be leveraged to respond to queries of many different kinds related to the patient journeys and physician preferences. As an example, the treatment assistance platform 304 may determine, based on the structured data described above, historical outcomes of patents that are treated with the treatment patterns identified in the insights and registry data 516. The data may also show how similar patients are treated by different physicians, for example. Thus, the data may explain why a physician gave one treatment instead of another treatment, and what the outcome of the different treatments were. Tangential to this information is the determination of a likelihood that a patient having certain characteristics, e.g., matching those of similar patients in the insights and registry data 516, will progress to a next stage of disease, have an adverse event, etc. Accordingly, the insights and registry data 516 generated by the pattern engine 518 and the machine intelligence engine 520 can be used to make predictions about how patients are likely to respond to various treatment options. Examples of how visualizations generated based on the insights and registry data 516 can be used to drive medical treatment decisions are described further below.

To further clarify the function of the treatment assistance platform 304 and the benefits that it can provide, several examples are presented here. The treatment assistance platform 304 can deduce, e.g., through a hypothesis of treatment patterns and additional knowledge of the physicians through repeated interactions, physician objective of a treatment decision. For example, whether certain treatment activities were taken to cure, control disease spread, improve quality of life for patient, etc. By going back in time and identifying sequence of events, the platform provides explainability of treatment blocks. For example, the platform can provide an explanation on physician belief/rationale for selecting a specific treatment. The platform can provide artificial intelligence (AI) assisted prediction on clinical pathways. In one embodiment, AI may be used to identify patients with similar disease conditions and how they are treated by other physicians. Based on those outcomes the platform can show one or more suggested clinical pathways. The platform can provide AI assisted recommendations on overall cost and quality of life for each of the suggested clinical pathways. In one embodiment, claims data has costs associated with each hospital visit. By identifying each clinical pathway, the platform can use AI to predict potential cost of treatment and hospitalization. The platform can cluster patients with similar co-morbidities and patient journeys.

The platform can provide insights and registry data 516 explaining other treatment pattern and treatment plan information. The data can explain roles and responsibilities of multiple physicians involved in treatment decisions of a patient. In an embodiment, several physicians are involved in a patient journey. Through the insights data, the platform can identify the key influencers, and their beliefs/biases about certain treatment plans.

The treatment assistance platform 304 can include a search engine 522 and a visualization engine 524. Together the engines can be used to search the insights and registry data 516 and generate interactive visualizations based on the insights and registry data 516 to respond to queries made through digital assistant applications 310 on client devices 306. The search engine 522 can be capable of searching the aggregated datasets of the treatment assistance platform 304 using natural language search, e.g., on taxonomical metadata 508 or insights and registry data 516. More particularly, the search engine 522 can search for both raw and derived metadata. The visualization engine 524 has the ability to visualize search results and aggregate details, e.g., at geographic level, individual patient level, physician level, payer level, and provider level. More particularly, the visualization engine 524 can provide use-case driven visualizations of raw data, derived data, and predictions. Accordingly, the digital assistant applications 310 can be used to perform searches in the treatment assistance platform 304 to act as recommenders that are used by the stakeholders of the ecosystem to obtain information that is relevant to them.

As an example of a use case, a payer 110 may use a digital assistant application 310 to request a recommendation or information about what to do for a cohort of patents at a health system level. Alternatively, a pharmaceutical representative may want to understand treatment patterns of a physician to understand whether the education that they have provided to the physician has helped influence the physician behavior. For example, at operation 408, the platform can send, in response to a search query including a name of a physician, the treatment pattern data 802. The treatment pattern data 802, which was developed by the pattern engine 518, as described above, can allow the representative to view how physician behavior has changed over time, and thus, allows many inferences to be made by the representative. For example, the representative can visualize whether the physician is trending toward using drugs that the representative has recommended. Examples of interactive visualizations are provided below.

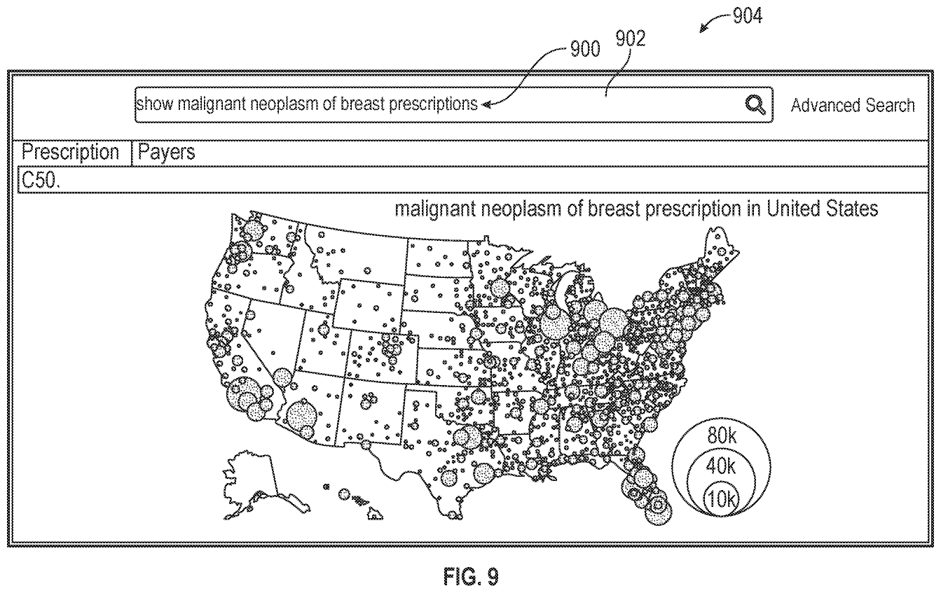

Referring to FIG. 9, a pictorial view of a user interface visualization of diagnosed patients at a national level is shown in accordance with an embodiment. The digital assistant application 310 can submit a search query 900 to search the treatment assistance platform 304 using a natural language search. For example, a user may input “show malignant neoplasm of breast prescription” into a search field 902 of a user interface 904. The computing system 302 can receive the search query 900 from the digital assistant application 310 executing on one of the remote client devices 306, search geocoded raw data, e.g., stored in the insights and registry data 516, and responsively transmit the responsive data to the visualization engine 524. The visualization engine 524, running on the computing system 302 or the remote client device 306 can then generate for display a visual model mapping malignant neoplasm of breast prescription in the United States, for example.

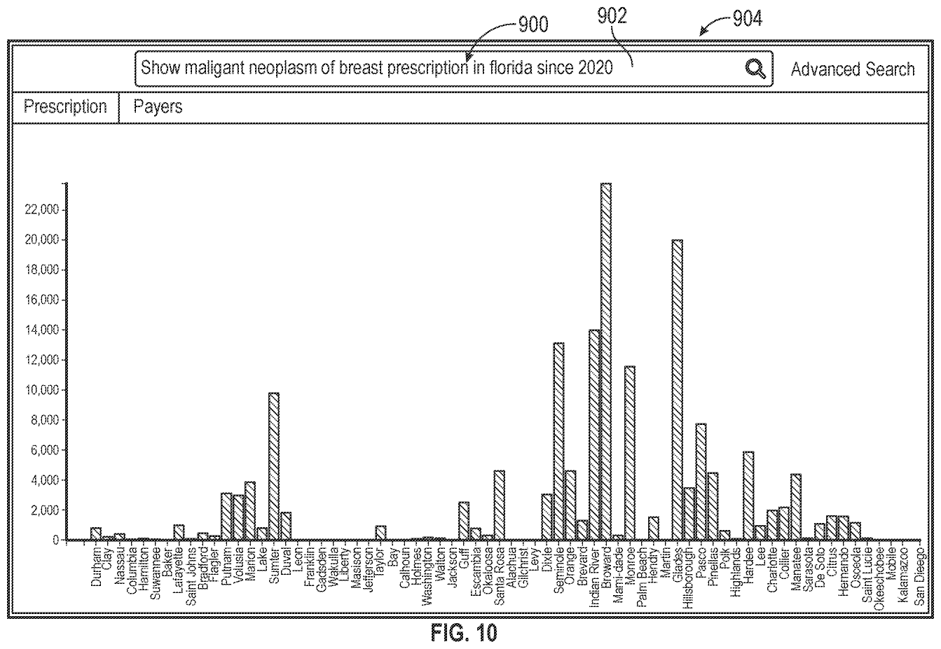

Referring to FIG. 10, a pictorial view of a user interface visualization of diagnosed patients at a state and local level is shown in accordance with an embodiment. The digital assistant application 310 can submit a search query 900 to search the treatment assistance platform 304 using a natural language search. For example, a user may input “show malignant neoplasm of breast prescriptions in florida since 2020” into the search field 902 of the user interface 904. The computing system 302 can receive the search query 900 from the digital assistant application 310 executing on one of the remote client devices 306, search geocoded raw data, e.g., stored in the insights and registry data 516, and responsively transmit the responsive data to the visualization engine 524. The visualization engine 524, running on the computing system 302 or the remote client device 306 can then generate for display a visual model charting malignant neoplasm of breast prescriptions in various Floridian cities during 2020, for example.

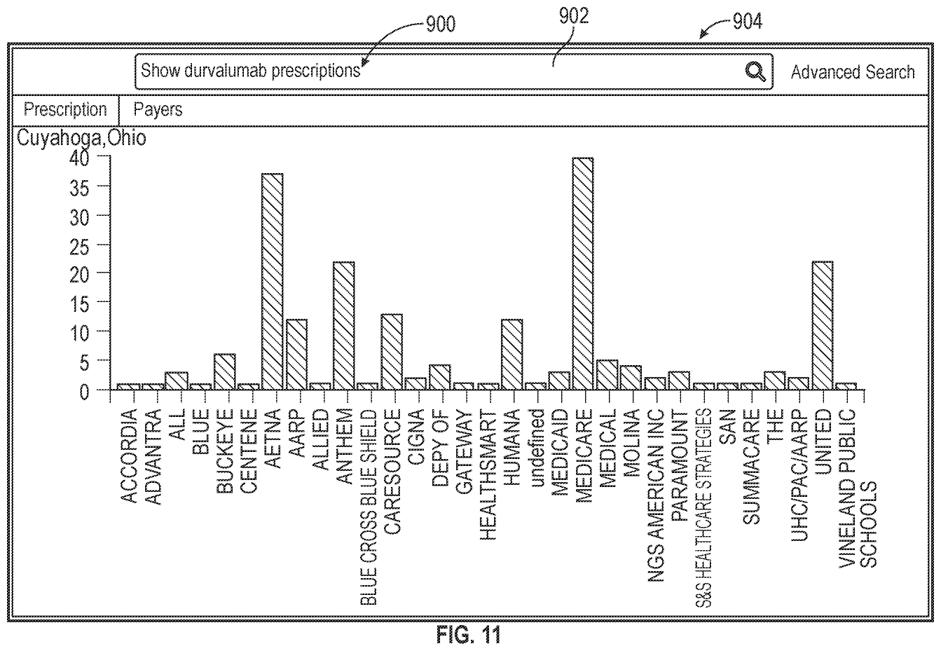

Referring to FIG. 11, a pictorial view of a user interface visualization of a payer view for diagnosed patients is shown in accordance with an embodiment. The digital assistant application 310 can submit a search query 900 to search the treatment assistance platform 304 using a natural language search. For example, a user may input “show durvalumab prescriptions” into the search field 902 of the user interface 904. The search may further include a target geography, e.g., Cuyahoga, Ohio. The computing system 302 can receive the search query 900 from the digital assistant application 310 executing on one of the remote client devices 306, search geocoded and/or taxonomical raw data, e.g., stored in the insights and registry data 516, and responsively transmit the responsive data to the visualization engine 524. The visualization engine 524, running on the computing system 302 or the remote client device 306 can then generate for display a visual model charting durvalumab prescriptions in the requested city, for example.

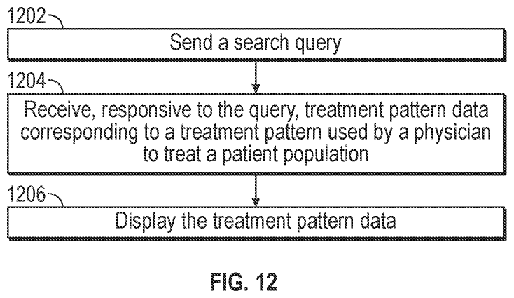

Referring to FIG. 12, a flowchart of a method of searching for visualizations to assist medical treatment decisions is shown in accordance with an embodiment. It will be appreciated from the several examples above that the treatment assistance platform 304 provides a powerful tool for users to visualize raw data, e.g., geocoded and taxonomical data collected from claims, EMR records, etc. The treatment assistance platform 304 can also be used to obtain derived data about, e.g., patient treatment plans and physician treatment patterns. As with the above examples, at operation 1202, a user may enter a search query 900, and the search query 900 can be sent from the digital assistant application 310 executing on a remote client device 306 to the computing system 302. For example, the search query 900 may include a physician name. The computing system 302 may perform the method described above with respect to operations 402-408, and transmit, in response to the search query 900 including the name of the physician, treatment pattern data 802 that was determined based on the structured data. Accordingly, at operation 1204, the digital assistant application 310 can receive, responsive to the search query 900, the treatment pattern data 802 corresponding to a treatment pattern used by the named physician. More particularly, the treatment pattern data 802 can describe a treatment pattern used by the physician to treat a patient population. As described below, at operation 1206, the received treatment pattern data 802 can be displayed, e.g., through the user interface 904 of the digital assistant application 310.

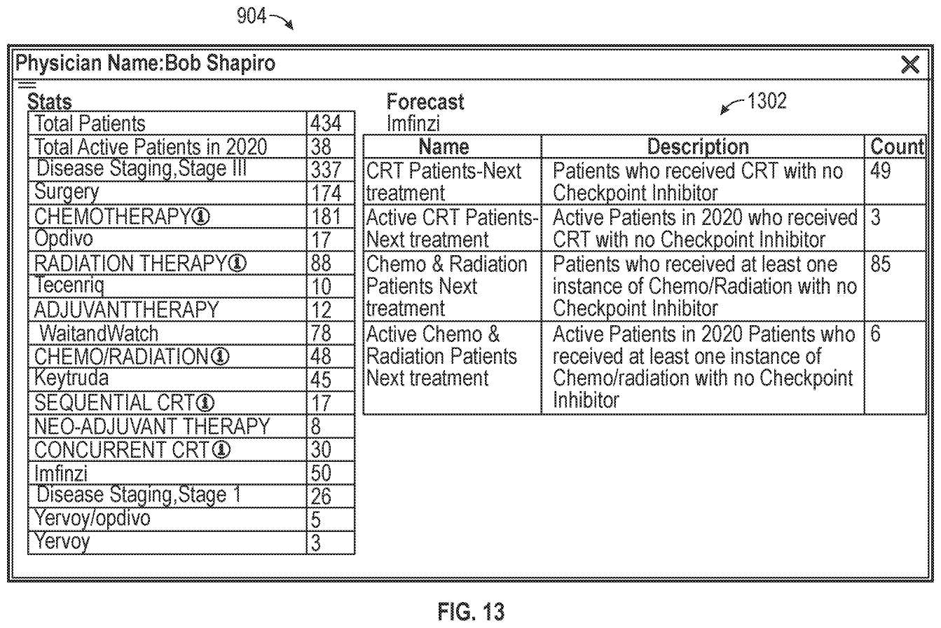

Referring to FIG. 13, a pictorial view of a physician summary view of a user interface is shown in accordance with an embodiment. The user interface 904 of the digital assistant application 310 can display the treatment pattern data 802 in a tabular form. For example, the named physician can have statistics associated with the treatment pattern, including a total number of patients, as well as segmented numbers of patients that have undergone particular treatment blocks, e.g., chemotherapy. This treatment pattern data 802 can be based on the treatment blocks that are derived by the pattern engine 518 and machine intelligence engine 520, as described above. Furthermore, predictive data 1302 can be included in the displayed information. The predictive data 1302 can provide information about a forecasted use of a particular drug for various patient segments. The forecast can be based on historical data that indicates that the physician has used the drug in similar situations before, and thus, is likely to do so again. When used by a representative of the pharmaceutical company 108, this information can be used to make recommendations to the physician that may be expected to align with the physician 104 beliefs, and thus, may be personalized to the physician.

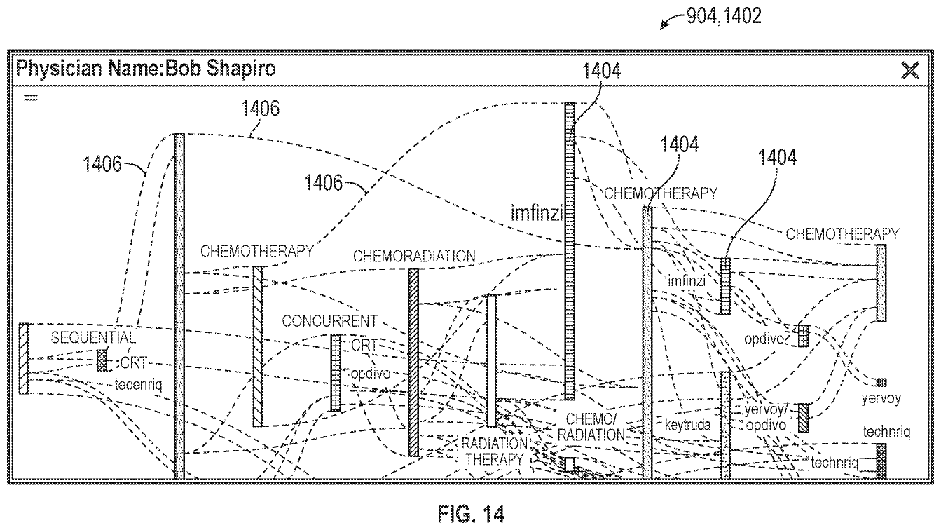

Referring to FIG. 14, a pictorial view of a treatment pattern view of a user interface is shown in accordance with an embodiment. The treatment pattern data 802 can be displayed in the user interface 904 on a display of the remote client device 306 as a model of the treatment pattern 1402. The model can include one or more treatment blocks 1404 distributed across the user interface 904. For example, each treatment block 1404, which represents a relevant treatment block 1404 from the insights and registry data 516, can be represented as vertical bands distributed chronologically from left to right. The treatment blocks 1404 may be interconnected by visual connectors 1406 to represent the chronological connection between the blocks, e.g., the sequence of treatment that the physician uses for the patient population.

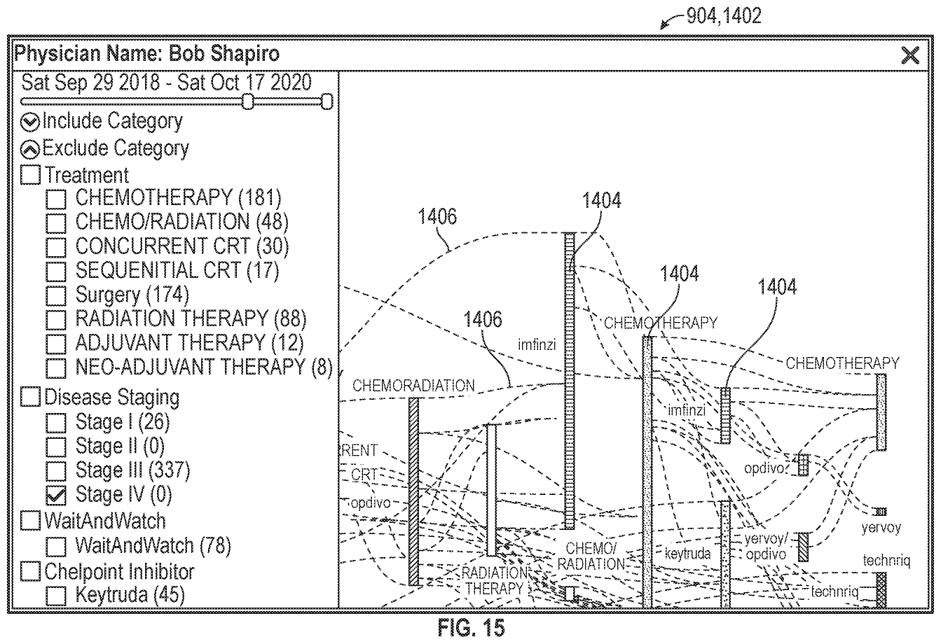

Referring to FIG. 15, a pictorial view of a patient funnel selector of a user interface is shown in accordance with an embodiment. The treatment pattern data 802 may be segmented to visualize how the physician approaches treatment of particular patient cohorts. In an embodiment, the search query 900 includes patient criteria similar to other patients treated with the treatment pattern 1402. For example, the user may check filter boxes in the user interface 904 to add categories into the search query 900 for inclusion or exclusion from the search results. In the illustrated example, the user has checked the “Stage IV” box for exclusion, and thus, the visualization will omit treatment patterns used for Stage IV cancer treatment patients. It will be appreciated that, if the user wished to review the treatment pattern 1402 typically used by the physician to treat Stage III cancer treatment patients, then the appropriate box could be checked to include that category. In response to the search query 900, the treatment assistance platform 304 could then transmit a treatment recommendation. For example, the visualization would then show treatment blocks 1404 that the physician uses in the case of Stage III cancer treatment patients, and that treatment pattern 1402 may be viewed as a treatment recommendation for a treatment that a particular patient, e.g., a Stage III cancer treatment patient, needs. It will be appreciated that the treatment would not only be particular to the patient, but also to the physician and the expected physician behavior.

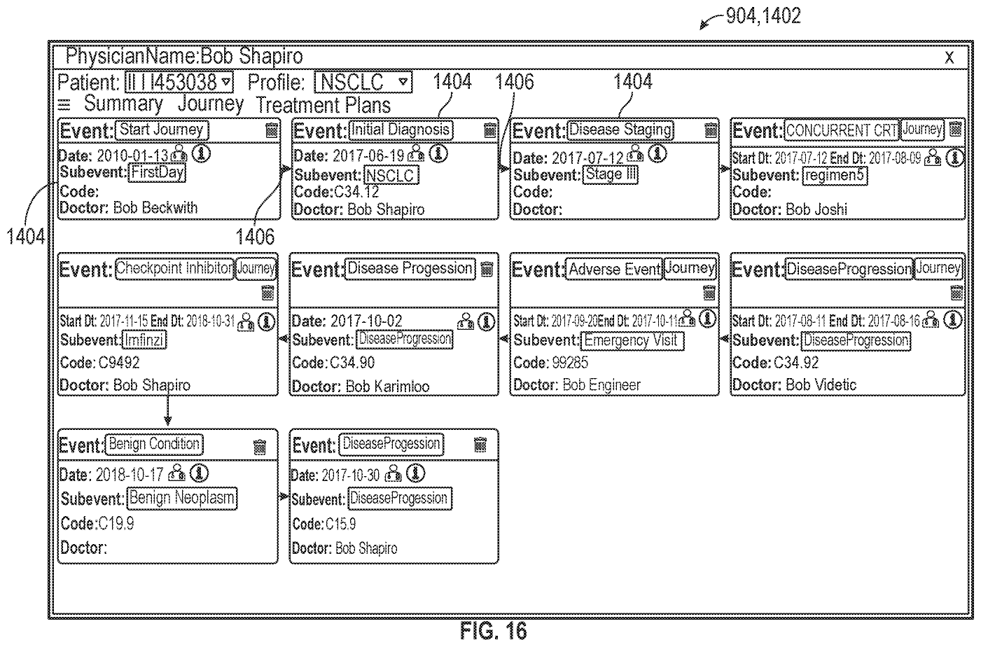

Referring to FIG. 16, a pictorial view of a treatment plan summary of a user interface is shown in accordance with an embodiment. The user interface 904 can display, in response to a search query 900 including a particular patient of the physician, the patient journey of the particular patient. The patient journey includes the treatment plan having treatment blocks 1404. As shown, the treatment blocks 1404 can be interconnected by visual connectors 1406 to indicate a chronological sequence of the treatment blocks 1404. Each treatment block 1404 can contain information about the treatment block 1404, e.g., event, data, subevent, code, and doctor information. The included information can be part of the raw data or derived data collected or generated by the treatment assistance platform 304. Accordingly, the user interface 904 can provide a full picture of the particular treatment plan used or suggested for the patient.

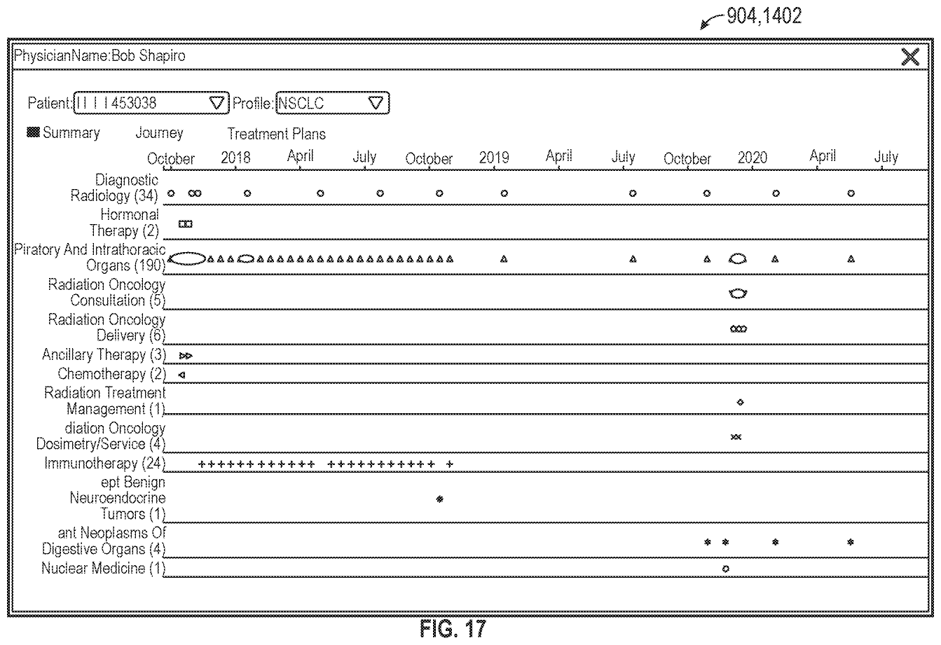

Referring to FIG. 17, a pictorial view of a patient journey summary of a user interface is shown in accordance with an embodiment. Another example of the patient journey summary is shown in which the user interface 904 includes, for the particular patient, treatment activity viewed on a chronological timetable. For example, treatment activity such as diagnostic radiology events, hormonal therapy events, ancillary therapy events, etc., can be represented as dots on the timetable. The timetable can include a full timeframe from the initiation of treatment, or may be filtered to only display certain time segments of the patient journey. Accordingly, the user interface 904 can provide a visualization of treatment activity over time.

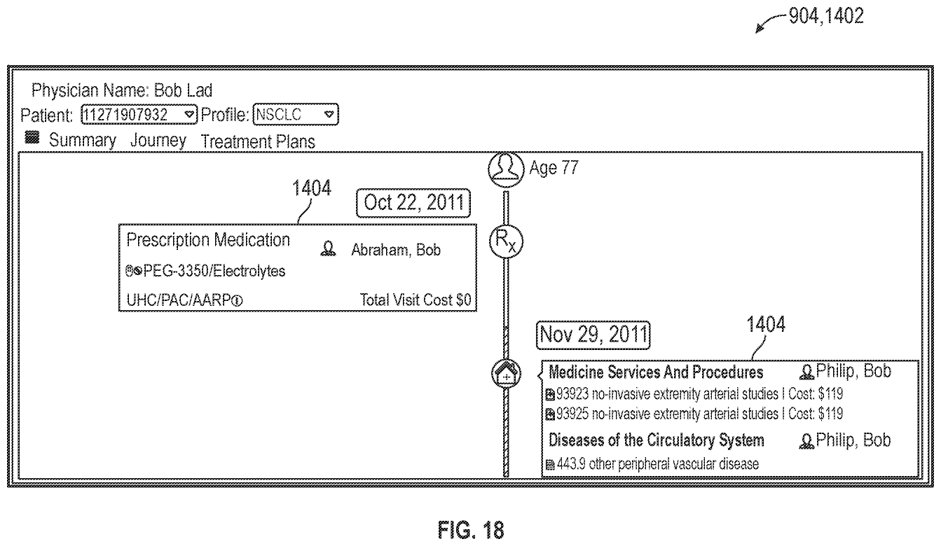

Referring to FIG. 18, a pictorial view of a patient journey timeline visualization of a user interface is shown in accordance with an embodiment. Another example of the patient journey summary is shown in which the user interface 904 allows the user to view treatment blocks 1404 on a timeline. The timeline can show treatment blocks 1404 along with representative dates of treatment activity. For example, a user can see that on or around Nov. 29, 2011, the particular patient was treated according to three claim codes.

Using any of the patient journey visualizations described above, a user such as a medical representative of the pharmaceutical company 108 can review and annotate patient journeys. For example, the user can make annotations that are captured as expert knowledge 506 (see FIG. 7 in which annotations are made using the user interface 904 of FIG. 18). Accordingly, the visualizations can be used by stakeholders to receive and view outputs of the treatment assistance platform 304 as well as to enter and provide inputs to the treatment assistance platform 304.

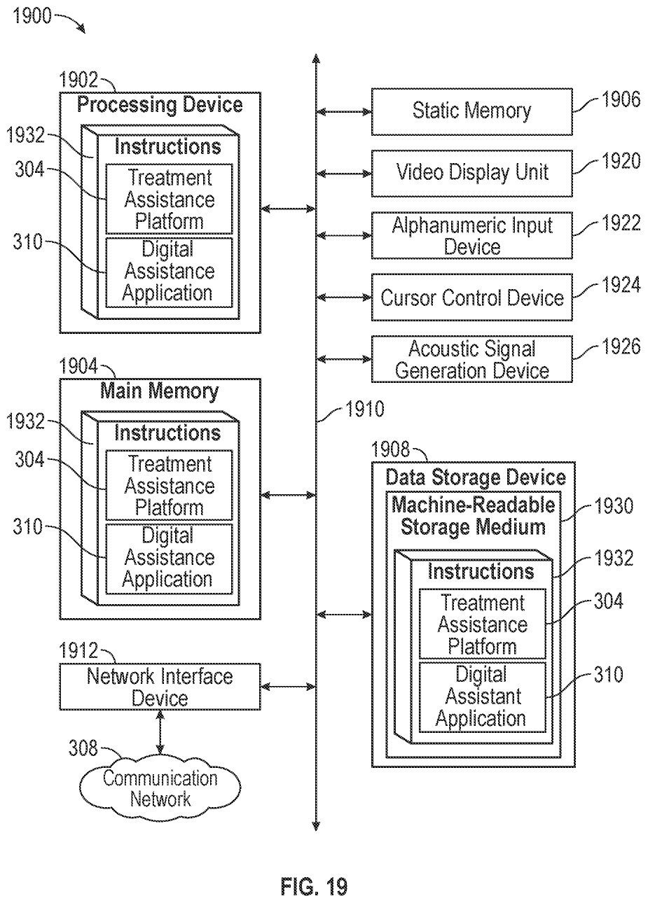

Referring to FIG. 19, a block diagram of an example computing device that may perform one or more of the operations described herein, according to some embodiments. Computing device 1900 may be connected to other computing devices in a LAN, an intranet, an extranet, and/or the Internet. The computing device may operate in the capacity of a server machine, e.g., computing system 302, or a client machine, e.g., remote client device 306, in a client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. In some embodiments, while only a single computing device is illustrated, the term “computing device” may be taken to include any collection of computing devices that individually or jointly execute a set (or multiple sets) of instructions to perform the methods discussed herein.

The example computing device 1900 may include a processing device (e.g., a general purpose processor, a PLD, etc.) 1902, a main memory 1904 (e.g., synchronous dynamic random access memory (DRAM), read-only memory (ROM)), a static memory 1906 (e.g., flash memory and a data storage device 1908), which may communicate with each other via a bus 1910.

Processing device 1902 may be provided by one or more general-purpose processing devices such as a microprocessor, central processing unit, or the like. In an illustrative example, processing device 1902 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 1902 may comprise one or more special-purpose processing devices such as an application specific integrated circuit (ASIC), a field programmable gate array (FPGA), a digital signal processor (DSP), network processor, or the like. The processing device 1902 may be configured to execute the operations described herein, in accordance with one or more aspects of the present disclosure, for performing the operations and steps discussed herein.

Computing device 1900 may include a network interface device 1912 which may communicate with a communication network 308. The computing device 1900 may include a video display unit 1920 (e.g., a liquid crystal display (LCD) or a cathode ray tube (CRT)), an alphanumeric input device 1922 (e.g., a keyboard), a cursor control device 1924 (e.g., a mouse) and an acoustic signal generation device 1926 (e.g., a speaker). In one embodiment, video display unit 1920, alphanumeric input device 1922, and cursor control device 1924 may be combined into a single component or device (e.g., an LCD touch screen).

Data storage device 1908 may include a non-transitory computer-readable storage medium 1930 on which may be stored one or more sets of instructions 1932 that may include instructions for one or more components (e.g., treatment assistance platform 304 or digital assistant application 310) for carrying out the operations described herein, in accordance with one or more aspects of the present disclosure. Instructions 1932 may reside, completely or at least partially, within main memory 1904 and/or within processing device 1902 during execution thereof by computing device 1900, main memory 1904 and processing device 1902 constituting computer-readable media. The instructions 1932 may be transmitted or received over the communication network 308 via network interface device 1912.

As described above, treatment decisions made by physicians can be multi-dimensional and complex. For example, a physician can prescribe a drug based on patient characteristics that are varied. The treatment assistance platform 304 can generate a treatment pattern for a physician. The treatment pattern can include treatments, e.g., drugs, that the physician historically prescribes for a situation. The platform 304 can identify the treatment, which is conditioned on the situation, and determine that the situational condition current exists. For example, the platform 304 can determine whether a target patient is currently at a stage of disease at which the physician usually prescribes the drug. The platform 304 can then provide that information to the physician to allow the physician to make the decision to prescribe the drug. For example, the platform 304 can recommend that the drug be prescribed to the target patient and support the recommendation with data showing the historical treatment pattern.

The treatment assistance platform 304 can not only recommend to the physician a next treatment, but can also predict how a patient will respond to the treatment. In an embodiment, the treatment assistance platform 304 can aggregate data to determine outcomes of a patient population (or a cohort of the patient population). A target patient can be identified, e.g., by inputting a patient identifier to the platform 304, and the platform can determine that the target patient is similar to the patient population (or cohort). In response to determining the similarity, the platform 304 can generate a prediction about an outcome of the target patient. The predicted outcome can be provided to a user of the digital assistant application 310, e.g., the physician, to allow the user to make treatment decisions. In an embodiment, the outcomes tracked in the historical data, and the outcomes predicted for the target patient, can be one or more of progression free survival, overall survival, hospitalizations, adverse events, or quality of life. Quality of life may be measured, for example, based on whether patients reported mood conditions, such as depression, after treatment. The data may be presented in a manner showing improvements in the outcomes when patient received the particular treatment versus outcomes of similar patients that received other treatments.

The treatment assistance platform 304 can generate physician treatment patterns and patient responses, as described above. The treatment pattern and patient response data can be aggregated to determine, for a patient cohort, data that can help decisions made by several users. For example, the platform 304 may aggregate treatment patterns of a group of physicians to be provided to a physician that is similar to the group. Similarity or fit of the physician in to the group of physicians can be determined, e.g., based similarities in treatment patterns. The physician may therefore be provided with data, e.g., treatment recommendations, according to the physician preferences.

Aggregated data can be targeted to other users, such as health systems and payers. For example, treatment patterns from a group of physicians within a health system, e.g., a hospital system, can be aggregated to determine a group treatment pattern. Such information can be used to guide the health system in making policies, guidelines, etc. As an example, outcome data targeted to the health systems can include hospitalization rates, which can be used by hospitals to decide the likelihood that they will be reimbursed by payers for using a particular treatment.

Data can be aggregated for use by payers. For example, data generated relative to physicians that have made treatments reimbursed by a particular payer can be aggregated and analyzed. The data analysis can provide information on standard treatment patterns that can help the payer determine policies and guidelines for treatment reimbursements. As an example, outcome data targeted to the payers can include per patient cost burden to allow payers to determine optimal treatment strategies that deserve reimbursement because they have been shown to effectively treat patients.

When determining similarities between physician and/or patient groups, information can be further segmented at a geographic or demographic level. For example, payers may use the platform 304 to determine treatment patterns of physicians practicing in a particular region or state, to help inform decisions about policies for that particular region or state.

To further clarify the description above, the information that is generated and shared by the platform 304 through the digital assistant applications 310 can be targeted to, received by, and used by any of the members of the medical treatment ecosystem 100. For example, the predictions and recommendations may be targeted directly to the physician to assist treatment decisions, targeted to representatives of the pharmaceutical company to facilitate appropriate interactions and conversations with the physician, or targeted to the health system or payer to allow appropriate guideline and policy setting. At this stage, it will be appreciated that such targeted information allows for the best outcomes for patients by empowering each of the ecosystem participants with the information that is most suitable to their purpose in serving the patients.

While computer-readable storage medium 1930 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” may be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

As used herein, the singular forms “a”, “an” and “the” are intended to include the plural forms as well, unless the context clearly indicates otherwise. The terms “comprises”, “comprising”, “includes”, and/or “including”, when used herein, may specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

In some embodiments, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A computer-implemented method, comprising:
receiving, by one or more processors of a computing system, unstructured data including real world data, scientific document data, and expert knowledge data corresponding to patient journeys;
generating, by the one or more processors based on the unstructured data, structured data including expert defined rules based on the expert knowledge data;
determining, by the one or more processors based on the structured data, treatment pattern data corresponding to a treatment pattern used by a physician to treat a patient population; and
transmitting, by the one or more processors of the computing system in response to a search query including a name of the physician, the treatment pattern data.

2. The computer-implemented method of claim 1 further comprising receiving, by the one or more processors, the search query from a digital assistant application executing on a remote client device.

3. The computer-implemented method of claim 1, wherein generating the structured data includes generating multi-category taxonomy data based on one or more of the real world data or the scientific document data.

4. The computer-implemented method of claim 1, wherein the expert knowledge data includes one or more of drug indication data or patient response data, wherein the drug indication data describes a disease that a drug is approved to treat, and wherein the patient response data describes how a patient is likely to respond to the drug.

5. The computer-implemented method of claim 1, wherein the treatment pattern includes one or more treatment blocks, and wherein each treatment block includes a respective sequence of treatment activity.

6. The computer-implemented method of claim 5 further comprising:
receiving, by a remote client device of the computing system, the treatment pattern data; and
displaying, in a user interface on a display of the remote client device, the treatment pattern, wherein the displayed treatment pattern includes the one or more treatment blocks interconnected by visual connectors.

7. The computer-implemented method of claim 1 further comprising:
determining, by the one or more processors based on the structured data, historical outcomes of patients treated with the treatment pattern; and
transmitting, by the one or more processors in response to the search query including patient criteria similar to the patients treated with the treatment pattern, a treatment recommendation.

8. A non-transitory computer readable medium storing instructions, which when executed by one or more processors of a computing system, cause the computing system to perform a method comprising:
receiving, by the one or more processors, unstructured data including real world data, scientific document data, and expert knowledge data corresponding to patient journeys;
generating, by the one or more processors based on the unstructured data, structured data including expert defined rules based on the expert knowledge data;
determining, by the one or more processors based on the structured data, treatment pattern data corresponding to a treatment pattern used by a physician to treat a patient population; and
transmitting, by the one or more processors in response to a search query including a name of the physician, the treatment pattern data.

9. The non-transitory computer readable medium of claim 8, wherein the method includes receiving, by the one or more processors, the search query from a digital assistant application executing on a remote client device.

10. The non-transitory computer readable medium of claim 8, wherein generating the structured data includes generating multi-category taxonomy data based on one or more of the real world data or the scientific document data.

11. The non-transitory computer readable medium of claim 8, wherein the expert knowledge data includes one or more of drug indication data or patient response data, wherein the drug indication data describes a disease that a drug is approved to treat, and wherein the patient response data describes how a patient is likely to respond to the drug.

12. The non-transitory computer readable medium of claim 8, wherein the treatment pattern includes one or more treatment blocks, and wherein each treatment block includes a respective sequence of treatment activity.

13. The non-transitory computer readable medium of claim 12, wherein the method includes:
receiving, by a remote client device of the computing system, the treatment pattern data; and
displaying, in a user interface on a display of the remote client device, the treatment pattern, wherein the displayed treatment pattern includes the one or more treatment blocks interconnected by visual connectors.

14. The non-transitory computer readable medium of claim 8 further comprising:
determining, by one or more processors based on the structured data, historical outcomes of patients treated with the treatment pattern; and
transmitting, by the one or more processors in response to the search query including patient criteria similar to the patients treated with the treatment pattern, a treatment recommendation.

15. A computing system, comprising:
a memory storing instructions; and
one or more processors configured to execute the instructions to cause the system to perform a method comprising:
receiving, unstructured data including real world data, scientific document data, and expert knowledge data corresponding to patient journeys,
generating, based on the unstructured data, structured data including expert defined rules based on the expert knowledge data,
determining, based on the structured data, treatment pattern data corresponding to a treatment pattern used by a physician to treat a patient population, and
transmitting, in response to a search query including a name of the physician, the treatment pattern data.

16. The computing system of claim 15, wherein the method includes receiving, by the one or more processors, the search query from a digital assistant application executing on a remote client device.

17. The computing system of claim 15, wherein generating the structured data includes generating multi-category taxonomy data based on one or more of the real world data or the scientific document data.

18. The computing system of claim 15, wherein the expert knowledge data includes one or more of drug indication data or patient response data, wherein the drug indication data describes a disease that a drug is approved to treat, and wherein the patient response data describes how a patient is likely to respond to the drug.

19. The computing system of claim 15, wherein the treatment pattern includes one or more treatment blocks, and wherein each treatment block includes a respective sequence of treatment activity.

20. The computing system of claim 15, wherein the method includes:
determining, by one or more processors based on the structured data, historical outcomes of patients treated with the treatment pattern, and
transmitting, by the one or more processors in response to the search query including patient criteria similar to the patients treated with the treatment pattern, a treatment recommendation.
